(19) United States
(12) Patent Application Publication
(10) Pub. No.: US 2022/0380295 A1
(43) Pub. Date: Dec. 1, 2022

(54) METHOD FOR SEPARATING AND PREPARING MULTIPLE ISOMER COMPOUNDS FROM SEABUCKTHORN POMACE

(71) Applicant: Northwest Institute of Plateau Biology, Chinese Academy of Sciences, Xining City (CN)

(72) Inventors: Na HU, Xining City (CN); Chenyu SU, Xining City (CN); Honglun WANG, Xining City (CN); Qi DONG, Xining City (CN)

(21) Appl. No.: 17/743,281

(22) Filed: May 12, 2022

(30) Foreign Application Priority Data
May 13, 2021 (CN) 202110523974.8

(51) Int. Cl. C07C 67/58; C07C 51/48

(52) U.S. Cl. CPC C07C 67/58; C07C 51/48; C07C 2603/52

(57) ABSTRACT

The invention provides a method for separating and preparing triterpenoid acids in seabuckthorn pomace, which mainly comprises extracting and purifying triterpenoid acids, and purifying and separating using a chromatographic method under specific conditions. The present invention effectively separates two groups of isomers of triterpene acid from seabuckthorn pomace simultaneously at the first time through specific methods and conditions, and provides possibility for further research and utilization of seabuckthorn pomace.

### CROSS REFERENCE TO RELATED APPLICATION

This application claims priority benefit of Chinese Patent Application No. 202110523974.8, filed on May 13, 2021, and the entire contents of which are incorporated herein by reference.

### TECHNICAL FIELD

The present invention relates to the field of natural plant chemistry.

### BACKGROUND

At present, most of our development, research and production of seabuckthorn products are concentrated in two parts of seabuckthorn fruits and seabuckthorn leaves, and as people pay more and more attention to health issues, development of health food has a broad market and outstanding potential. At present, seabuckthorn fruits are mainly used in production of juice, wine and seabuckthorn leaves are mainly used in production of medicine and tea. According to reports, in my country, there are millions of tons of seabuckthorn pomace after seabuckthorn industry produces seabuckthorn products every year. If we do not pay more attention to it and do not carry out comprehensive utilization, it will cause a lot of wastes of natural product resources. Therefore, full utilization of seabuckthorn pomace has become a new research hotspot. Improving research and production of active ingredients of seabuckthorn pomace is conducive to promoting deep utilization of seabuckthorn pomace and improving added value of seabuckthorn food. It is of great significance in turning waste into treasure, saving energy and reducing emissions, and promoting economy.

The inventors found that the seabuckthorn pomace contains a variety of triterpenoid acids in the early stage, and the experiments have shown that the triterpenoid acids extracted from the seabuckthorn pomace have good liver protection and other effects, and have development value of health care product. However, deeper research on monomeric compounds is still in progress.

### SUMMARY

In order to better conduct in-depth research on active components in seabuckthorn pomace, the present invention intends to provide a method for separating and preparing triterpenoids in seabuckthorn pomace.

The inventor found in the research that seabuckthorn pomace contains a variety of isomers of triterpene acids, however, because the isomers are very similar in structure, it is difficult to separate and detect.

The present invention finally provides a method for separating and preparing 4 isomers from seabuckthorn pomace through trial and error, which comprises:

(1) taking seabuckthorn pomace, extracting with alcohol and then precipitating with water, loading the precipitate on a C18 column, and eluting with 20%±5, 60%±5, and 80%±5 of aqueous methanol solutions and pure methanol in turn, and collecting a first yellow band eluted with pure methanol;

(2) loading the yellow band on C18 column to perform chromatography with isocratic elution of an aqueous methanol solutions of 80-90:20-10 v/v and a flow rate of 14-18 mL/min, and collecting an eluent at 29-50 min, and removing a solvent to obtain a fraction F4-2;

(3) loading the fraction F4-2 on C18 column to perform chromatography with an elution procedure of: A-water, B-acetonitrile, 65%-72% B at 0-17.5 min, 72%-73% B at 17.5-20 min, 73%-77% B at 20-40 min, 77%-100% B at 40-60 min, and a flow rate of 14-18 mL/min; collecting 4th to 7th chromatographic peaks in turn from 7 obvious strong peaks; removing a solvent, and obtaining compounds 3-6 with the following structures, respectively:

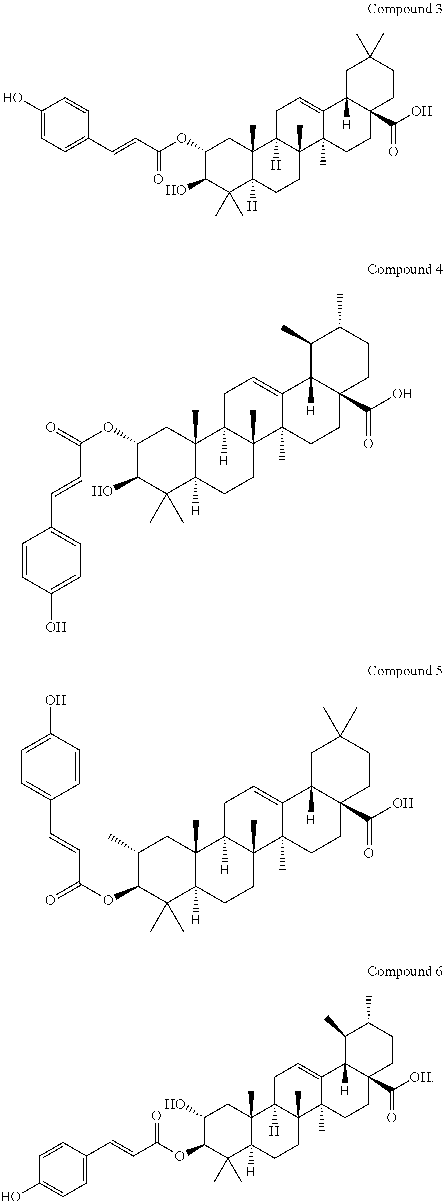

Wherein, in step (1), 85% v/v or more of ethanol is used for the extracting with alcohol and the precipitating with water.

Further, in step (1), 95% v/v or more of ethanol is used for the extracting with alcohol and the precipitating with water.

Wherein, during performing chromatography in steps (2) and (3), a detection wavelength is 210 nm.

Further, the chromatographic column used in step (2) is selected from Xaqua C18, 250×21.2 mm, 5 μm.

Further, the chromatographic column used in step (3) is selected from Kromasil C18, 250×21.2 mm, 5 μm.

Further, in step (2), the isocratic elution of aqueous methanol solutions of 85:15 v/v is adopted.

Further, in steps (2) and (3), the elution flow rate is 16 mL/min.

The present invention also provides a method for separating and preparing 2 isomers from seabuckthorn pomace, which comprises:

(1) taking seabuckthorn pomace, extracting with alcohol and then precipitating with water, loading the precipitate on a C18 column, and eluting with 20%±5, 60%±5, and 80%±5 of aqueous methanol solutions and pure methanol in turn and collecting a first yellow band eluted with pure methanol;

(2) loading the yellow band on C18 column to perform chromatography with isocratic elution of a aqueous methanol solutions of 80-90:20-10 v/v and a flow rate of 14-18 mL/min, and collecting an eluent at 29-50 min, and removing a solvent to obtain a fraction F4-2;

(3) loading the fraction F4-2 on C18 column to perform chromatography with an elution procedure of: A-water, B-acetonitrile, 65%-72% B at 0-17.5 min, 72%-73% B at 17.5-20 min, 73%-77% B at 20-40 min, 77%-100% B at 40-60 min, and a flow rate of 14-18 mL/min; collecting 2^{th} to 3^{th} chromatographic peaks in turn from 7 obvious strong peaks; removing a solvent, and obtaining compounds 1 and 2 with the following structures, respectively:

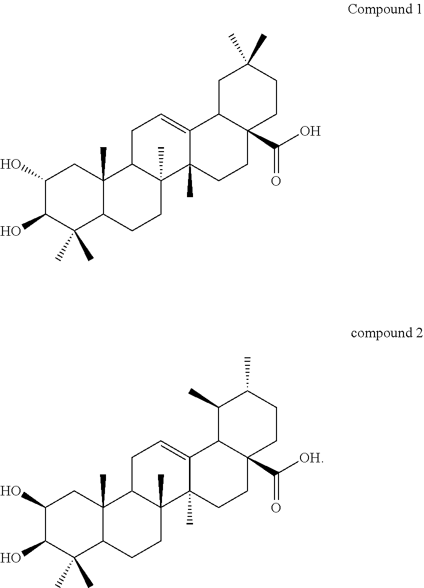

The present invention also provides a method for separating and preparing 6 isomers from seabuckthorn pomace, which comprises:

(1) taking seabuckthorn pomace, extracting with alcohol and then precipitating with water, loading the precipitate on a C18 column, and eluting with 20%±5, 60%±5, and 80%±5 of aqueous methanol solutions and pure methanol in turn and collecting a first yellow band eluted with pure methanol;

(2) loading the yellow band on C18 column to perform chromatography with isocratic elution of a aqueous methanol solutions of 80-90:20-10 v/v and a flow rate of 14-18 mL/min, and collecting an eluent at 29-50 min, and removing a solvent to obtain a fraction F4-2;

(3) loading the fraction F4-2 on C18 column to perform chromatography with an elution procedure of: A-water, B-acetonitrile, 65%-72% B at 0-17.5 min, 72%-73% B at 17.5-20 min, 73%-77% B at 20-40 min, 77%-100% B at 40-60 min, and a flow rate of 14-18 mL/min; collecting 2^{th} to 7^{th} chromatographic peaks in turn from 7 obvious strong peaks; removing a solvent, and obtaining compounds 1-6 with the following structures, respectively:

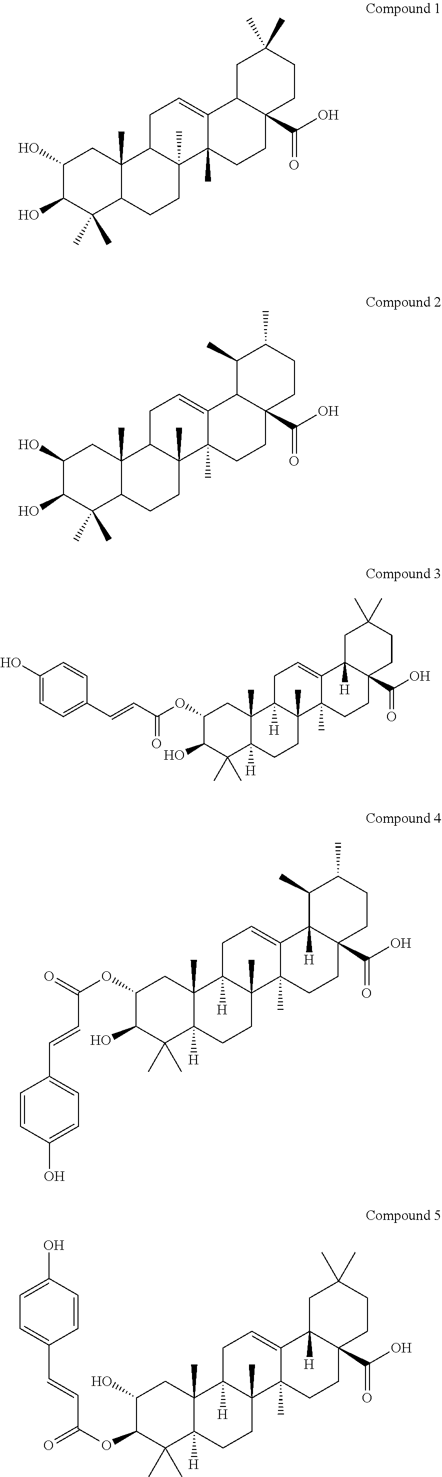

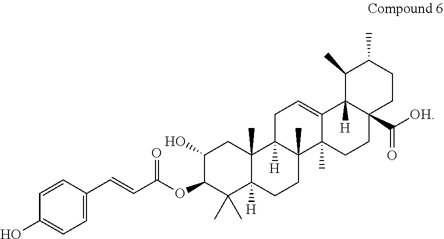

The present invention effectively separates two groups of isomers of triterpene acids from seabuckthorn pomace simultaneously at the first time through specific methods and conditions, and provides possibility for further research and utilization of sea buckthorn pomace.

### BRIEF DESCRIPTION OF THE DRAWINGS

FIG. 1 illustrates fractions F4 and F5 eluted by pure methanol.

FIG. 2 illustrates HPLC chromatogram of the fraction F4 from seabuckthorn pomace.

FIG. 3 illustrates HPLC chromatogram of the fraction F4-2 from seabuckthorn pomace.

FIG. 4 illustrates chromatogram under comparative condition 1.

FIG. 5 illustrates chromatogram under comparative condition 2.

### DETAILED DESCRIPTION

The present invention is further explained below in combination with specific embodiments, but the embodiments do not limit the present invention in any form.

### Example 1

Extraction of Triterpenoid Acids from Seabuckthorn Pomace

The dried seabuckthorn pomace was crushed, added with 95% of ethanol at the ratio of material to liquid of 1:10 kg/L, heated and refluxed at a temperature of 70° C. for 1.5 h, 3 times, and filtered. The filtrate was pooled and concentrated by rotary evaporation at 60° C. to remove the solvent in order to obtain an ethanol extract from seabuckthorn pomace, which was frozen at −20° C.

Enrichment of Triterpene Acids from Seabuckthorn Pomace

Primary treatment of triterpenoid acids from seabuckthorn pomace: 1.0 kg of seabuckthorn extract was weighed, added with 2.0 L of distilled water, and heated in a water bath at 60° C. until the extract was dissolved, and stands still for precipitation, and filtered and separated to obtain a filter residue, which is dried and pulverized to obtain a crude extract of triterpene acids.

Preparation of triterpenic acids by ODS under a reduced pressure: 6.20 g of the dried crude extract was weighted, added with an appropriate amount of methanol to prepare a suspension, mixed with the dry ODS filler in a mass ratio of 1:1.5 (g/g) and then dried.

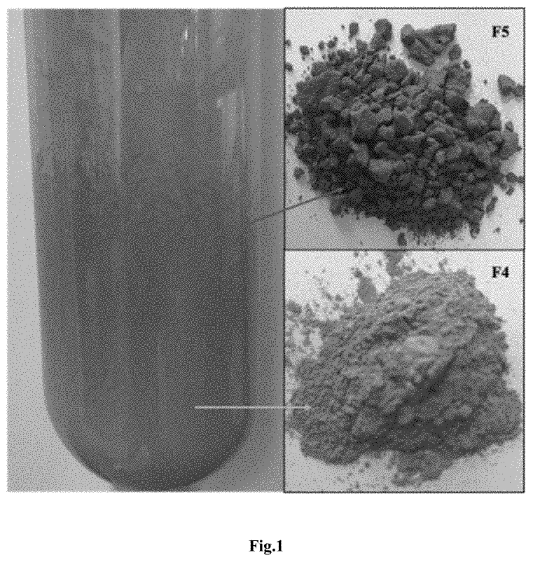

3-5 BV of 20% methanol in water (v/v), 60% methanol in water (v/v), 80% methanol in water (v/v), and pure methanol was used successively for gradient elution. The fractions eluted with 20%, 60%, and 80% of methanol were named as F1, F2, and F3 successively. The fraction eluted with methanol was divided into two bands (see FIG. 1), the first yellow band was named as F4 and the second red band was named as F5.

(3) 0.1 g of F4 dry powder was formulated into a solution with a concentration of 50 mg/mL in methanol, centrifuged to obtain a supernatant. The supernatant was filtered with a filter membrane of 0.45 μm.

Chromatographic column: Xaqua C18 (250×21.2 mm, 5 μm); mobile phase: a methanol-water solution (85:15, v/v), isocratic elution; elution time: 80 min; flow rate: 16 mL/min; detection wavelength: 210 nm.

According to peak times of compounds, the peak time of fraction F4-1 is 0-20 min, the peak time of fraction F4-2 is 29-50 min, and the peak time of fraction F4-3 is 60-70 min. The collected fractions were centrifugated, concentrated and dried for the next step of separation.

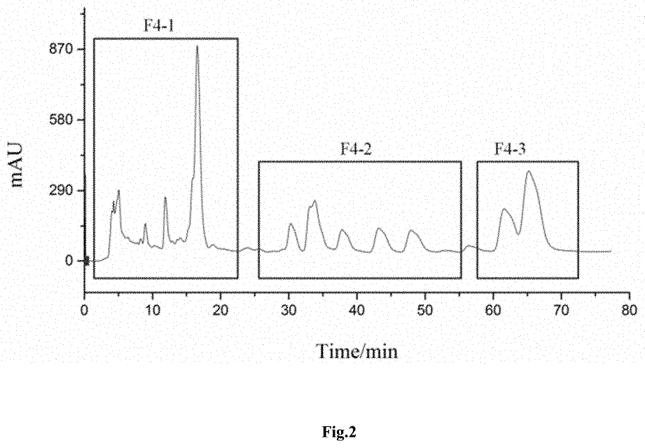

The prepared spectrum was shown in FIG. 2.

(4) The fraction F4-2 was purified and separated by the following method:

Column: Kromasil C18 (250×21.2 mm, 5 μm); an elution program: A-water, B-acetonitrile, 65%-72% B at 0-17.5 min, 72%-73% B at 17.5-20 min, 73%-77% B at 20-40 min, 77%-100% B at 40-60 min; elution time: 50 min; flow rate: 16 mL/min; detection wavelength: 210 nm.

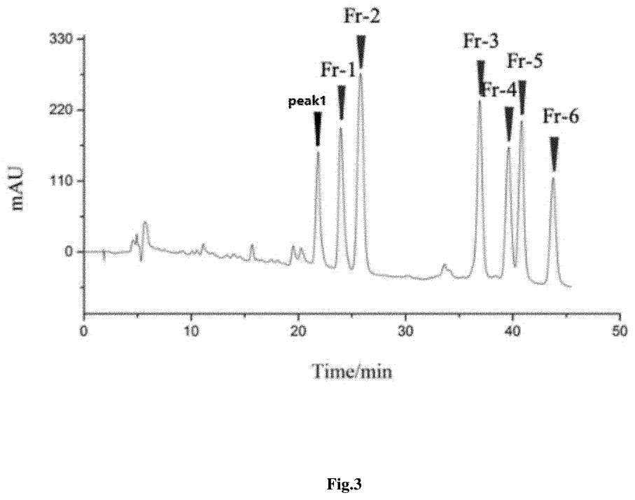

The prepared chromatogram of the fraction F4-2 was shown in FIG. 3. There were 7 distinct chromatographic peaks in total, and the main compounds can achieve baseline separation. By this method, a total of 6 monomer compounds of Fr-1, Fr-2, Fr-3, Fr-4, Fr-5 and Fr-6 were obtained.

Compound 1: Maslinic acid, a white amorphous powder, chemical formula: C₃₀H₄₈O₄.

¹H-NMR data (600 MHz, MeOD, δ, ppm): 5.28 (t, J=3.30 Hz, H-12); 3.95 (dt, J1=10.32 Hz, J2=4.36 and 4.32 Hz, H-2); 3.50 (d, J=9.36 Hz, H-18); 3.25 (dd, J1=13.68 Hz, J2=4.24 Hz, H-3); 3.10 (dd, J1=7.58 Hz, J2=11.64 Hz, H-2); 2.85 (td, J1=14.10 Hz, J2=4.14 Hz, H-18); 2.30 (s, H-3); 1.17 (s, Me-27); 1.08 (s, Me-23); 0.94 (s, Me-25); 0.92 (s, Me-30); 0.90 (s, Me-29); 0.86 (s, Me-24); 0.84 (s, Me-26). ¹³C-NMR data (600 MHz, MeOD, δ, ppm): 181.8 (C-28); 145.3 (C-13); 123.5 (C-12); 84.5 (C-3); 69.5 (C-2); 56.7 (C-5) 48.1 (C-9); 48.5 (C-1); 47.2 (C-17); 47.6 (C-19); 42.7 (C-14); 42.9 (C-18); 40.5 (C-8); 40.6 (C-4); 39.3 (C-10); 34.9 (C-21); 33.8 (C-22); 33.9 (C-29); 33.6 (C-7); 31.6 (C-20); 29.3 (C-23); 28.8 (C-15); 26.4 (C-27); 24.1 (C-11); 24.0 (C-30); 24.6 (C-16); 19.6 (C-6); 17.5 (C-26); 17.1 (C-25); 17.7 (C-24). The compound was determined to be maslinic acid by comparing with the literature data and the standard substance.

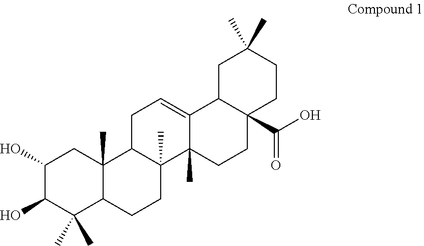

Compound 2: corosolic acid, a white amorphous powder, chemical formula: C₃₀H₄₈O₄.

¹H-NMR data (600 MHz, MeOD, δ, ppm): 5.28 (t, J=3.30 Hz, H-12); 3.61 (1H, m), 3.30 (dd, J1=7.58 Hz, J2=11.64 Hz, H-2); 2.92 (1H, d, J=9.5 Hz), 2.20 (d, J1=14.10 Hz, J2=4.14 Hz, H-18); 2.00 (s, H-3), 1.62 (m, H-3), 1.38 (1H, m), 1.10 (s, Me-27), 1.03 (s, Me-23), 0.98 (s, Me-25), 0.88 (s, Me-30), 0.85 (s, Me-29), 0.81 (s, Me-24), 0.76 (s, Me-26); ¹³C-NMR data (600 MHz, MeOD, δ, ppm): δC 181.6 (C-28), 139.8 (C-13), 126.7 (C-12), 84.5 (C-3), 69.5 (C-2), 55.8 (C-5), 54.4 (C-18), 49.5 (C-17), 48.3 (C-1, C-9), 40.5 (C-4), 56.7 (C-5), 40.8 (C-19), 40.4 (C-20), 43.3 (C-8, C-14), 39.2 (C-10), 34.2 (C-7), 38.1 (C-22), 29.3 (C-15), 31.8 (C-21), 29.2 (C-23), 24.5 (C-27), 17.5 (C-29), 24.1 (C-11), 25.3 (C-16), 19.5 (C-6), 21.6 (C-30), 17.8 (C-26), 17.6 (C-25), 17.2 (C-24). The compound was determined to be corosolic acid by comparing with the literature data and the standard substance.

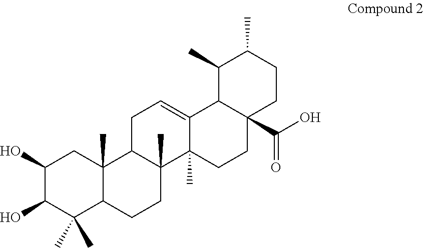

Compound 3: 2-O-trans-p-coumaroyl maslinic acid, a white amorphous powder, chemical formula: C39145406.

¹H NMR (600 MHz, MeOD, δ, ppm): 7.65 (1H, d, J=16.1, H-7′), 7.45 (1H, d, J=8.4, H-2′, 6′), 6.88 (1H, d, J=8.4, H-3′, 5′), 6.73 (1H, d, J=16.1, H-8′), 5.26 (1H, m, H-12), 4.58 (1H, d, J=9.8, H-3), 3.79 (1H, m, H-3), 2.87 (1H, dd, J=14.0, 4.2, H-18), 1.30 (3H, s, H-27), 1.18 (3H, s, H-25), 1.03 (3H, s, H-24), 0.95 (3H, s, H-30), 0.91 (3H, s, H-29), 0.88 (3H, s, H-23), 0.83 (3H, s, H-26); ¹³C NMR (600 MHz, MeOD, δ, ppm): 181.9 (C-28), 169.8 (C-9′), 159.3 (C-4′), 145.1 (C-7′), 144.0 (C-13), 133.1 (C-2′, 6′), 127.5 (C-1′), 122.5 (C-12), 116.8 (C-3′, 5′), 115.1 (C-8′), 84.6 (C-3), 66.9 (C-2), 55.8 (C-5), 51.1 (C-9), 49.2 (C-1), 48.4 (C-17), 47.2 (C-19), 47.2 (C-14), 46.8 (C-18), 42.3 (C-4), 42.2 (C-8), 34.5 (C-10), 33.3 (C-21), 32.5 (C-7), 31.2 (C-22), 30.1 (C-29), 29.9 (C-20), 29.4 (C-23), 29.0 (C-15), 26.6 (C-27), 24.8 (C-11), 24.2 (C-16), 24.1 (C-30), 19.6 (C-6), 18.5 (C-24), 17.9 (C-26), 17.2 (C-25). The compound was determined to be 2-O-trans-p-coumaroyl maslinic acid by comparing with the literature data and the standard substance.

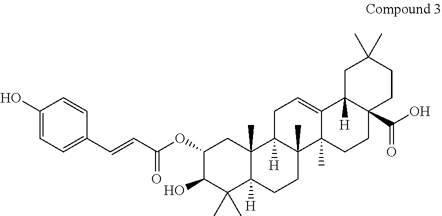

Compound 4: 2-O-trans-p-coumaroyl corosolic acid, a white amorphous powder, chemical formula: C39145406.

¹H NMR (600 MHz, MeOD, δ, ppm): 7.64 (1H, d, J=16.1, H-7′), 7.46 (1H, d, J=8.4, H-2′, 6′), 6.87 (1H, d, J=8.4, H-3′, 5′), 6.72 (1H, d, J=16.1, H-8′), 5.24 (1H, m, H-12), 4.59 (1H, d, J=9.8, H-3), 3.80 (1H, m, H-3), 2.23 (1H, d, J=11.9, H-18), 1.14 (3H, s, H-27), 1.05 (3H, s, H-25), 0.97 (3H, d, J=6.3, H-30), 0.95 (3H, s, H-24), 0.89 (3H, s, H-23), 0.88 (3H, d, J=6.3, H-29), 0.83 (3H, s, H-26); ¹³C NMR (600 MHz, MeOD, δ, ppm): 181.9 (C-28), 169.8 (C-9′), 159.3 (C-4′), 145.1 (C-7′), 144.0 (C-13), 133.1 (C-2′, 6′), 127.5 (C-1′), 122.5 (C-12), 116.8 (C-3′, 5′), 115.1 (C-8′), 84.6 (C-3), 66.9 (C-2), 55.8 (C-5), 51.1 (C-9), 49.2 (C-1), 48.4 (C-17), 47.2 (C-19), 47.2 (C-14), 46.8 (C-18), 42.3 (C-4), 42.2 (C-8), 34.5 (C-10), 33.3 (C-21), 32.5 (C-7), 31.2 (C-22), 30.1 (C-29), 29.9 (C-20), 29.4 (C-23), 29.0 (C-15), 26.6 (C-27), 24.8 (C-11), 24.2 (C-16), 21.7 (C-30), 19.6 (C-6), 18.6 (C-24), 17.9 (C-26), 17.8 (C-29), 17.4 (C-25). The compound was determined to be 2-O-trans-p-coumaroyl corosolic acid by comparing with the literature data and the standard substance.

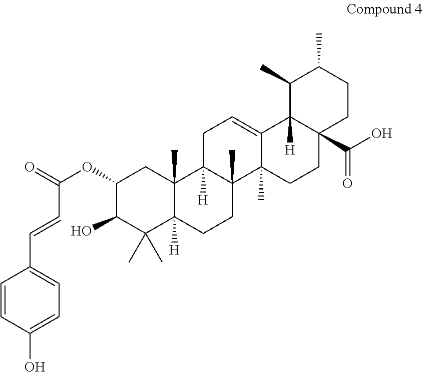

Compound 5: 3-O-trans-p-coumaroyl maslinic acid, a white amorphous powder, chemical formula: C39145406.

¹H-NMR (600 MHz, MeOD, δ, ppm) δ: 7.63 (1H, d, J=15.9 Hz, H-7′), 7.47 (2H, d, J=8.2 Hz, H-2′, 6′), 6.90 (2H, d, J=8.4 Hz, H-3′, 5′), 6.38 (1H, d, J=15.8 Hz, H-8′), 5.25 (1H, br s, H-12), 4.63 (1H, d, J=9.8 Hz, H-3), 3.84 (1H, m, H-2); ¹³C-NMR (600 MHz, MeOD, δ, ppm) δ: 180.7 (C-28), 168.3 (C-39), 161.7 (C-34), 145.3 (C-37), 145.1 (C-13), 131.0 (C-32, 36), 126.6 (C-31), 122.6 (C-12), 117.2 (C-33, 35), 116.5 (C-38), 85.4 (C-3), 66.7 (C-2), 55.9 (C-5), 50.8 (C-9), 49.1 (C-1), 47.8 (C-19), 47.7 (C-17), 47.6 (C-14), 47.4 (C-18), 47.3 (C-8), 47.1 (C-4), 38.8 (C-10), 34.6 (C-21), 33.6 (C-22, 29), 33.4 (C-7), 31. 3 (C-20), 29.3 (C-23), 28. 6 (C-15), 26.6 (C-27), 24.3 (C-16), 24.1 (C-30), 24.0 (C-11), 19.0 (C-6), 18.6 (C-26), 17.8 (C-25), 17.1 (C-24). The compound was determined to be 3-O-trans-p-coumaroyl maslinic acid by comparing with the literature data and the standard substance.

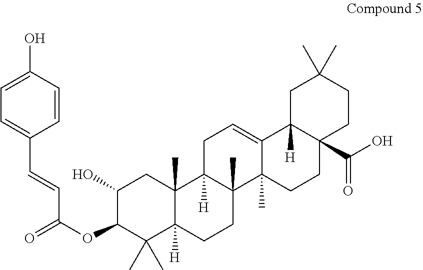

Compound 6: 3β-O-trans-p-coumaroyloxy-2a-hydroxyurs-12-en-28-oic acid, a white amorphous powder, chemical formula: C₃₉H₅₄O₆.

¹H-NMR data (600 MHz, MeOD, δ, ppm): 7.64 (2H, d, J=8.6 Hz, H-2′, 6′), 7.61 (1H, d, J=16.8 Hz, H-7′), 6.91 (2H, d, J=8.6 Hz, H-3′, 5′), 6.38 (1H, d, J=16.8 Hz, H-8′), 5.23 (1H, t, J=4.6 Hz, H-12), 4.63 (1H, d, J=10.2 Hz, H-3), 3.94 (1H, m, H-2), 3.3 (1H, m, H-18), 1.15 (3H, s, H-27), 1.06 (3H, s, H-25), 0.96 (6H, s, H-24, 30), 0.95 (3H, s, H-29), 0.89 (3H, s, H-23), 0.82 (3H, s, H-26); ¹³C-NMR (600 MHz, MeOD, δ, ppm): 180.3 (C-28), 167.5 (C-9′), 160.8 (C-4′), 144.0 (C-7′), 139.7 (C-13), 134.1 (C-2′, 6′), 127.0 (C-1′), 125.7 (C-12), 117.4 (C-8′), 116.2 (C-3′, 5′), 85.3 (C-3), 66.6 (C-2), 55.9 (C-5), 53.8 (C-18), 50.6 (C-1), 49.0 (C-17), 47.9 (C-9), 47.7 (C-14), 47.6 (C-21), 47.4 (C-8), 47.3 (C-4), 39.8 (C-19, 20), 38.6 (C-10), 37.8 (C-22), 33.7 (C-7), 29.4 (C-23), 29.0 (C-15), 25.2 (C-16), 24.3 (C-27), 24.0 (C-11), 21.8 (C-30), 19.0 (C-6), 18. 6 (C-26), 17.9 (C-29), 17.8 (C-25), 17.2 (C-24). The compound was determined to be 3β-O-trans-p-coumaroyloxy-2a-hydroxyurs-12-en-28-oic acid by comparing with the literature data.

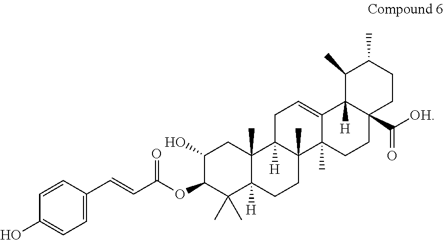

The inventor's experiments found that the separation of the compounds in the fraction F4-2 was relatively difficult. After many selections, 7 main chromatographic peaks were finally separated through specific chromatographic conditions, and 6 main compounds among them were identified. The following conditions cannot be used for better separation of the compounds in the fraction F4-2.

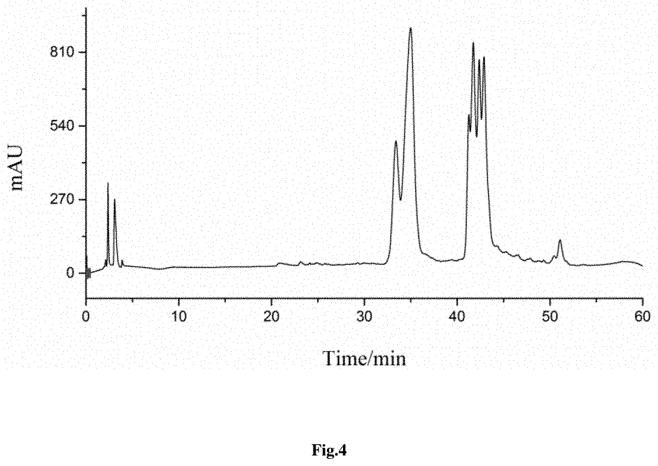

Comparative condition 1: Column: Xaqua C18 (250×4.6 mm, 5 μm); mobile phase: A-water, B-acetonitrile, gradient elution: 40%-100% B at 0-50 min; analysis time: 50 min; flow rate: 1 mL/min; detection wavelength: 210 nm; column temperature: 25° C. The chromatogram was shown in FIG. 4.

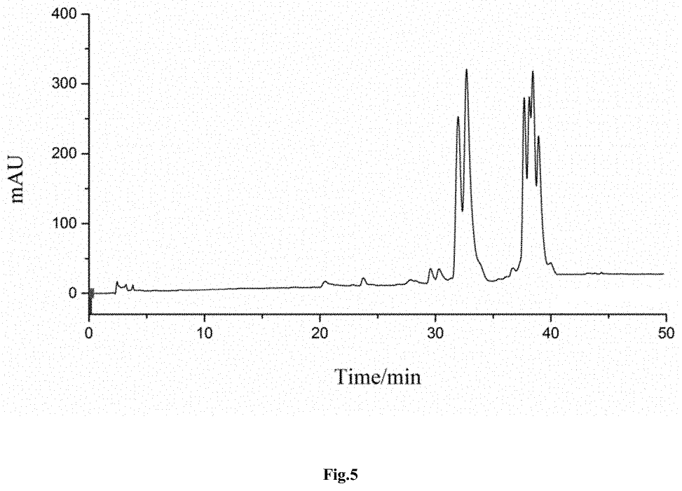

Comparative condition 2: Column: Xcharge C18 (250×4.6 mm, 5 μm); mobile phase: A-water, B-acetonitrile, a gradient elution: 0-50 min 40%-100% B; analysis time: 50 min; flow rate: 1 mL/min; detection wavelength: 210 nm; column temperature: 25° C. The chromatogram was shown in FIG. 5.

What is claimed is:

1. A method for separating and preparing 4 isomers from seabuckthorn pomace, characterized in that, the method comprises:
(1) taking seabuckthorn pomace, extracting with alcohol and then precipitating with water, loading the precipitate on a C18 column, and eluting with 20%±5, 60%±5, and 80%±5 of aqueous methanol solutions and pure methanol in turn, and collecting a first yellow band eluted with pure methanol;
(2) loading the yellow band on C18 column to perform chromatography with isocratic elution of an aqueous methanol solutions of 80-90:20-10 v/v and a flow rate of 14-18 mL/min, and collecting an eluent at 29-50 min, and removing a solvent to obtain a fraction F4-2;
(3) loading the fraction F4-2 on C18 column to perform chromatography with an elution procedure of: A-water, B-acetonitrile, 65%-72% B at 0-17.5 min, 72%-73% B at 17.5-20 min, 73%-77% B at 20-40 min, 77%-100% B at 40-60 min, and a flow rate of 14-18 mL/min; collecting 4th to 7th chromatographic peaks in turn from 7 obvious strong peaks; removing a solvent, and obtaining compounds 3-6 with the following structures, respectively:

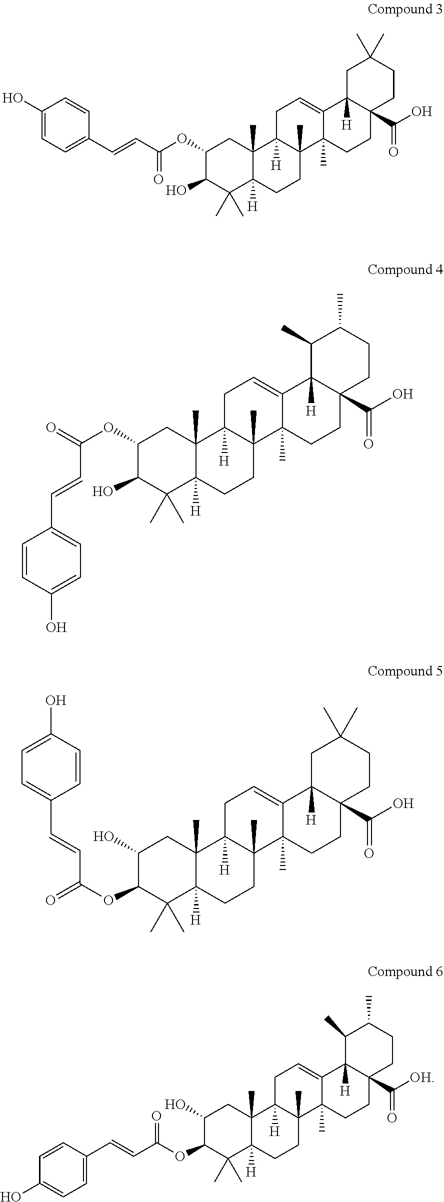

2. The method according to claim 1, characterized in that: in step (1), 85% v/v or more of ethanol is used for the extracting with alcohol and the precipitating with water.

3. The method according to claim 1, characterized in that: in step (1), 95% v/v or more of ethanol is used for the extracting with alcohol and the precipitating with water.

4. The method according to claim 1, characterized in that: during performing chromatography in steps (2) and (3), a detection wavelength is 210 nm.

5. The method according to claim 1, characterized in that: the chromatographic column used in step (2) is selected from Xaqua C18, 250×21.2 mm, 5 μm.

6. The method according to claim 1, wherein the chromatographic column used in step (3) is selected from Kromasil C18, 250×21.2 mm, 5 μm.

7. The method according to claim 1, wherein in step (2), the isocratic elution of aqueous methanol solutions of 85:15 v/v is adopted.

8. The method according to claim 1, wherein in steps (2) and (3), the elution flow rate is 16 mL/min.

9. A method for separating and preparing 2 isomers from seabuckthorn pomace, characterized in that, the method comprises:
(1) taking seabuckthorn pomace, extracting with alcohol and then precipitating with water, loading the precipitate on a C18 column, and eluting with 20%±5, 60%±5, and 80%±5 of aqueous methanol solutions and pure methanol in turn and collecting a first yellow band eluted with pure methanol;
(2) loading the yellow band on C18 column to perform chromatography with isocratic elution of an aqueous methanol solutions of 80-90:20-10 v/v and a flow rate of 14-18 mL/min, and collecting an eluent at 29-50 min, and removing a solvent to obtain a fraction F4-2;
(3) loading the fraction F4-2 on C18 column to perform chromatography with an elution procedure of: A-water, B-acetonitrile, 65%-72% B at 0-17.5 min, 72%-73% B at 17.5-20 min, 73%-77% B at 20-40 min, 77%-100% B at 40-60 min, and a flow rate of 14-18 mL/min; collecting 2^{th} to 3^{th} chromatographic peaks in turn from 7 obvious strong peaks; removing a solvent, and obtaining compounds 1 and 2 with the following structures, respectively:

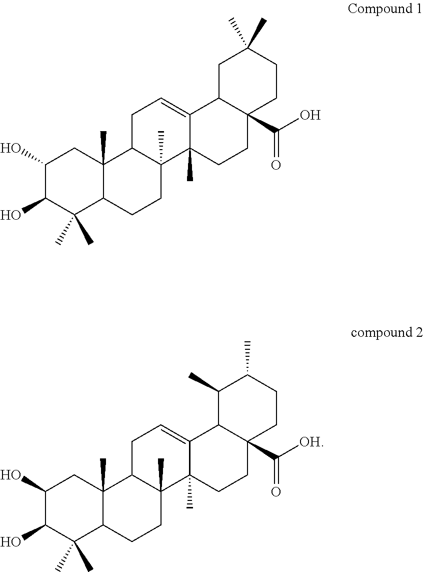

10. A method for separating and preparing 6 isomers from seabuckthorn pomace, characterized in that, the method comprises:
(1) taking seabuckthorn pomace, extracting with alcohol and then precipitating with water, loading the precipitate on a C18 column, and eluting with 20%±5, 60%±5, and 80%±5 of aqueous methanol solutions and pure methanol in turn and collecting a first yellow band eluted with pure methanol;
(2) loading the yellow band on C18 column to perform chromatography with isocratic elution of a aqueous methanol solutions of 80-90:20-10 v/v and a flow rate of 14-18 mL/min, and collecting an eluent at 29-50 min, and removing a solvent to obtain a fraction F4-2;
(3) loading the fraction F4-2 on C18 column to perform chromatography with an elution procedure of: A-water, B-acetonitrile, 65%-72% B at 0-17.5 min, 72%-73% B at 17.5-20 min, 73%-77% B at 20-40 min, 77%-100% B at 40-60 min, and a flow rate of 14-18 mL/min; collecting 2^{th} to 7^{th} chromatographic peaks in turn from 7 obvious strong peaks; removing a solvent, and obtaining compounds 1-6 with the following structures, respectively: [chemical structure]
